(19) United States
(12) Patent Application Publication
(10) Pub. No.: US 2009/0305335 A1
(43) Pub. Date: Dec. 10, 2009

(54) Method for analysing a sample

(75) Inventors: Ari Kuusisto, Turku (FI); Lahja Kolehmainen-Seveus, Danderyd (SE)

Correspondence Address: BUCHANAN, INGERSOLL & ROONEY PC, POST OFFICE BOX 1404, ALEXANDRIA, VA, 22313-1404, US

(73) Assignee: Wallac Oy, Turku (FI)

(21) Appl. No.: 11/794,682

(22) PCT Filed: Jan. 4, 2006
(86) PCT No.: PCT/FI06/50008

§ 371 Date: Aug. 6, 2009

(30) Foreign Application Priority Data
Jan. 5, 2005 (FI) 20055007

(51) Int. Cl. C12Q 1/24; C12Q 1/04

(52) U.S. Cl. 435/30; 435/34

(57) ABSTRACT

This invention relates a method for analysing a biological sample by imaging, wherein sample processing and detection and optionally pre-treatment is performed in a single container. The invention further relates to a kit for use in said method.

### FIELD OF THE INVENTION

The present invention relates to a method for preparing and analysing a biological sample for imaging purposes in a single container. The invention also relates to a test kit for use in such a method.

### BACKGROUND OF THE INVENTION

In the clinics, there is a tendency to use rapid and simple tests (e.g. ‘dip stick’-tests) for analysing biological samples as such tests are often cheap and they are easily automated. However, not all biological samples can be analysed by such tests. Therefore, methods for analysing biological samples by microscopy or other means of imaging remain important since such methods give more reliable and accurate information on the specimen e.g. reliable identification of various cell populations. Data concerning a desired target can be combined with e.g. structural information. Moreover, visualization allows characterization of the presence or absence of a target in individual cells.

Conventional preparation of cell suspensions for microscopy is performed in test tubes and/or on glass microscope slides. Sample preparation in test tubes involves repeated cycles of centrifugation and subsequent removal of the supernatant. As the supernatant is discharged either by pipeting or by vacuum suction, the treatment inevitably results in a loss of cells. Some cell types may be more easily lost than others, which leads to distortions in the results. Moreover, the percentage of lost cells may vary from time to time. Sometimes repeated centrifugation steps may result in lowered level of specific signal or alter structural properties of the cells. Prior to the microscopic analysis, the sample has to be transferred onto a microscope slide by e.g. cytocentrifugation incurring further loss of cells. Alternatively, all or some of the sample preparation steps can be performed on said slide. Such procedures involve dipping and rinsing the slides in various solution chambers. Some cells may be more detach-prone than others. Between the various rinsing steps, excess solution may have to be discarded by wiping the slide. Such a treatment may damage the cells.

On the whole, conventional sample preparation for imaging purposes is laborious, time consuming, and require well-trained laboratory personnel.

US patent publication No. 20010055776 discloses the utilization of filter plates for assaying marker proteins in cell culture. Such filter plates allow removal of incubation solutions without exhausting centrifugation steps and they are well suited for automation. One of the disadvantages associated with the disclosed method is, however, that the sample is analysed by DELFIA time-resolved fluorometry measuring total light emission from the whole sample. Thus no information on localization of the labelled protein is obtained and it is not possible to get any information on the amount of the protein in an individual cell.

A special problem arises when a biological sample to be analysed contains various cell types but only some of the cell types are relevant for the analysis in question. Such mixed samples include e.g. body fluids, lavage fluids, faeces and especially sputum samples. Conventional microscopy analysis of such samples is complicated, requires manual processing and special skills as identification of the cells is based on the colour differences after cytological staining.

Another problem associated with some biological samples is that they are poorly preserved and should be processed immediately after being obtained from a subject. This applies specially to sputum samples that remain preserved only for two hours after sampling. Thus, the need for an automated sample preparation and analysis method is increased even further.

Sputum samples are mixed biological cell suspensions containing eosinophils, neutrophils, lymphocytes, basophils, macrophages and also epithelial cells originating from lungs and oral cavity. Diagnosis and monitoring of therapy of various respiratory diseases is based on the identification and counting of different cell types in sputum. A standard method for such identification consists of conventional sample preparation using cytological stains such as May-Grunwald-Giemsa (MGG) and manual microscopic analysis performed by a microscopist. Distinction of various cell types is based on the morphology of the cells. Thus, the main effort in conventional sample preparation is to preserve the physical appearance of the cells intact.

Immunoassays have been introduced to augment sputum analysis. One of the current methods for estimating the number of eosinophils in a sputum sample is based on detection of Eosinophilic Cationic Protein (ECP), a protein secreted by eosinophils upon their activation. It is generally assumed that the amount of ECP correlates with the number of eosinophils. The accuracy of the method is greatly reduced by the fact that eosinophils may contain varying amounts of EPC. Thus, simple quantification of ECP does not provide reliable results enough for sophisticated purposes such as disease diagnosis.

Metso et al. (Thorax 2002; 57:449-451) describes a method for analysing sputum samples, wherein eosinophils and neutrophils are identified using cell specific marker molecules. These are made visible using enzyme-anti-enzyme complexes and their substrates and a stain. This procedure requires several rinsing and drying steps and is thus both time-consuming and laborious. This method allows combining the results from antibody labelling (e.g. ECP) and structural parameters of the cells. However, this method is performed conventionally on a microscope slide.

Soini et al. (Microsc. Res. Tech. 2003; 62:396-407) discloses use of luminophores for multi-parameter imaging of different leukocyte types in peripheral blood. Also this method is performed conventionally on a microscope slide.

Thus, there is a need in the art for an accurate method for analysing mixed biological cell suspensions, which method should be easily automated. Especially there is a need for a fast method wherein the whole sample preparation procedure and the analysis would be automated. Moreover, the method should have the advantages of conventional microscopy.

US patent publication No. 20030228705 discloses an apparatus and a method suited for counting cellular elements in biological cell suspensions, wherein sample preparation and analysis is performed in a single apparatus equipped with a collection membrane. This method can be automated and it resolves some of the problems associated with sample preparation and analysis of biological cell suspensions. However, only one parameter in a sample is analysed and thus, all cells comprising that particular feature are visualized. This kind of one-parameter analysis is not suitable for mixed biological samples such as sputum as more than one parameter has to be analysed for reliable distinction of various cell types.

Thus, the need for a method for analysing mixed biological samples remains in the art.

### BRIEF DESCRIPTION OF THE INVENTION

The present invention is based on the surprising finding that mixed biological samples can be processed and imaged in a single container, such as a plate provided with a filter.

The present invention thus relates to a method for analysing a mixed biological sample by imaging, comprising the steps of a) pre-treating a sample obtained from a subject; b) transferring said sample into a container; c) processing said sample; d) detecting at least one target in said sample by imaging; and e) analysing more than one image, wherein steps c)-d) are performed in a single container. Optionally, step a) is also performed in said container and step b) is performed prior to step a).

More specifically the invention relates a method wherein said container is a multi-well device equipped with at least one filter.

The present invention further relates to a method wherein said mixed biological sample comprises granulocytes, inflammatory cells, cancer cells, cells from immunodefence system and/or foetal cells or said sample is a body fluid, a lavage fluid, a tissue homogenate, an expectoration sample, an excretion, an aspiration sample, a biopsy sample or a cell culture.

The present invention further relates to a kit for use in said method. In one embodiment, the kit comprises a container equipped with a means for draining liquid while retaining at least some of the cells in said sample and at least one labelling reagent for detecting a target. Optionally, the kit may further comprise other necessary reagents for performing pre-treatment and processing of said sample. In another embodiment, said container is a multi-well plate.

### BRIEF DESCRIPTION OF THE DRAWINGS

FIG. 1A is an image showing all the nuclei in a human leukocyte sample processed and imaged as described in Example 1.

FIG. 1B is an image of eosinophils in a leukocyte sample processed and imaged as described in Example 1.

### DETAILED DESCRIPTION OF THE INVENTION

The present invention relates to a method for analysing mixed biological samples comprising the following steps: transferring a sample into a vial or equivalent container optionally containing necessary reagents such as EDTA and citrate immediately after being obtained from a subject, pre-treating the sample, providing the sample into a container equipped with a means for collecting and draining, processing the specimen, labelling the target, detecting the target by imaging and analysing the images.

By “mixed biological sample” is herein meant any cell suspension, body fluid, lavage, expectoration sample, excretion sample, or any cell suspension from aspiration biopsy sample or other biopsy sample or tissue homogenate derived from an animal or human subject containing more than one cell population or subtype of cells. Typically, cell populations of interest include inflammatory cells (e.g. granulocytes, mast cells and basophils), cancer cells, cells from immuno-defense system (e.g. lymphocytes) and foetal cells. Body fluid samples include e.g. blood, urine, lacrimal, peritoneal and pleural fluids, whereas lavage samples include e.g. bronchoalveolar (BAL), nasal, effusion from middle ear, and cervical and intestinal samples. Expectoration samples include e.g. sputum whereas excretion samples include e.g. faeces. Aspiration biopsies can be derived from e.g. eye, breast tissue, bone marrow and umbilical cord whereas homogenized biopsy samples can be derived from any tissue or organ. Samples derived from cell cultures are included regardless of the origin of the cells. Suitable sources of cell suspension samples according to the invention are not limited to the examples described above. The terms “sample” and “specimen” are herein interchangeable.

For the purposes of the present invention, the term “target” is intended to include any biological subject such as a cell, a cell compartment (e.g. cytoplasm or a nucleus), an organelle (e.g. a granule, a lysosome, a mitochondrion or Golgi apparatus), a chromosome, a gene sequence, a molecule (e.g. a marker molecule, a cell-surface or intracellular antigen), a protein molecule (e.g. a hormone), a drug molecule, a carbohydrate (e.g. sugar residues on glycoproteins), a nucleic acid (e.g. DNA, RNA, viral DNA or viral RNA), a viral or bacterial particle, or a chemical element (e.g. Ca, K, Na) or any other similar particle (e.g. a microbead).

The term “parameter” is herein meant to include any desired feature in a sample such as the type, the size, the number, the shape, or the granularity of a target or a reference target. By “reference target” is meant any biological subject to which the desired target is compared to.

In the first step of the method according to the present invention (a), biological sample obtained from a human or animal subject is pre-treated. Pre-treatment may include mixing the sample with agents needed for protection or preservation of the targets. Suitable agents such as EDTA and citrate may be provided in a sample vial or they may be added after the sample has been transferred into the vial. Mixing the sample with the agents has to be performed promptly in order to avoid that the cells respond to an altered environment and possibly excrete the targets. Pre-treatment may also include removing a certain part of the sample. For instance, mucus may be removed from the sample by dithiotreitol (e.g. Sputolysin™) or other suitable reagent such as an enzyme known in the art. Sometimes it may be appropriate to remove a certain cell population from the sample, which can be achieved e.g. by lyzing the said cells. Partial removal of cells or other particles contained in the sample may also be achieved by using sieves, such as passing the sample through a filter having a predetermined pore size, thus removing some of the cells. Depending on the sample, pre-treatment may also include homogenization of the sample or organel extraction. Other optional pre-treatments are well known to persons skilled in the art.

In the second step of the invention (b), the sample is provided in a container equipped with a means for collecting the cells of interest and draining excess liquid and/or particles (e.g. cells) under a predetermined size as described in the US patent publications No. 20010055776 and No. 20030228705. Optionally, said container may contain more than one means for collecting and draining and it may be connected to waste or to another container so that drained material can be recovered. Features of the container and the means for draining depend on the nature of the sample and the labelling method chosen. Such features can be easily appreciated by a man skilled in the art.

In one preferred embodiment, said container is a multi-well device, and said means for draining is a filter.

In another particularly preferred embodiment, said device is a multi-well filter plate such as a 6-, 12-, 24-, 48-, 96-, 384- or 1536-well plate, or any other plate having a multi-well format. Multi-well filter plates are commercially available from several providers.

The third step of the method according to the present invention (c) is “sample preparation” which herein means any appropriate procedure(s) prerequisite for the analysis in question, and which are performed after providing the sample into the container. Sample preparation thus includes, but is not restricted to, fixation, stabilization, permeabilization, washing, blocking of non-specific targets, labelling and post-treatment, which are well known in the art. The terms “sample preparation” and “sample processing” are herein interchangeable.

Incubation or washing solutions may be removed from the container by aspiration through the means for draining after every sample preparation step. The aspiration may be obtained by means of a vacuum and it should be adjusted not to affect the cell structures as well known in the art.

In one embodiment according to the present invention, the biological sample is treated in such a way that morphology of the cells is preserved and antigenicity of the target(s) is retained. In the case of intracellular targets, cell membranes have to be permeabilized to allow entrance of labelling agents into the cell interior. Sometimes blocking of non-specific targets may be required. These sample preparation procedures are well known in the art and they are easily chosen depending on the sample and target in question.

Permeabilizing fixatives offer superior preservation of the granulocyte structure, immobilize the low molecular weight marker antigens, allow efficient penetration of the antibody conjugates into the cells, and retain the immunoreactivity of the markers. Thus, permeabilizing fixatives are well suited for use with samples containing neutrophils and/or eosinophils, that is e.g. blood, sputum and BAL. Preferable permeabilizing fixatives comprise aldehyde and at least one detergent. One especially preferable permeabilizing fixative is Ortho Permeafix™ (OPF).

For the purposes of the present invention, the term “labelling” is intended to mean any appropriate procedure that enables detection of the target. Such procedures include labelling the target directly with labelled bioaffinity probes or indirectly using primary bioaffinity probes against the target and secondary labelled bioaffinity probes against the primary bioaffinity probes. Suitable labels for use in the present invention include, but are not restricted to, stains directly binding to the target, luminophore, fluorochrome, enzyme, isotope or metal labelled bioaffinity probes (e.g. antibodies, antibody fragments, gene sequences, peptides, lectins), as well as biotinylated bioaffinity probes combined to luminophore, fluorochrome, enzyme, metal or isotope labelled streptavidin or streptavidin-biotin complexes. Different labelling methods may be co-applied in any desired combination to label or mark more than one target in a single cell or specimen.

In one preferred embodiment of the invention, long decay time photoluminescent labels, such as fluorescent lanthanide chelates and/or phophorescent metalloporphyrins are used for labelling. Suitable lanthanide chelates include terbium, samarium, dystrosium and europium, whereas suitable metalloporphyrins include palladium coproporphyrin and platinum coproporphyrin. Said labels may be conjugated to ligands such as antibodies. Preferably, for the best possible separation of one signal from the other signals and the background fluorescence (the disturbing fluorescence emission), fluorescent labels having a different decay time than the fluorescence originating from the other parts of the sample or the container or the instrument or the other labels but the said label are used.

The specimen may be post-fixed to enable storage. For long term storage (days or more) the specimen may be fixed with strongly gross-linking fixatives. For instance, glutaraldehyde is a preferable reagent for this purpose while other suitable reagents are well known in the art. Next, specimens are either dried or mounted with suitable agents such as permanent organic solvent based mounting media e.g. Merckoglas™ (Merck, Darmstadt, Germany) or any other suitable agents known in the art. Mounting enables long-term storage of the labelled samples and also results in better quality of the samples for imaging purposes.

The sample preparation procedures depend on the nature of the sample and the target to be analysed and are well known to skilled persons in the art. Said procedures can be performed only partially, in any suitable combination or in any suitable order. Optionally, pre-treatment may be performed in the same container as sample preparation and analysis. If desired, some sample preparation steps may be performed conventionally e.g. in a test tube. Incubation times and temperatures dependent on the particular protocol employed as well known in the art. The invention is not limited to the examples of sample preparation steps described above.

The fourth step of the method according to the present invention (d) is detecting the labelled target by imaging. Depending on the labelling method used, there are a number of options as to the best way to image the specimens. The most common instrument used is the bright field microscope, which is particularly useful for observing materials labelled with alkaline phosphatase, peroxidase or radiolabels. The latter labels can also be viewed by dark field microscopy. A fluorescence microscope is the obvious choice for observing specimens with fluorescent labels. Digital imaging systems are being used more frequently because of their high resolution, ability to detect signals that cannot be seen by conventional microscopes, and because they allow image processing and quantitative measurements. Different microscopy techniques such as reflection contrast microscopy, dark field microscopy, and phase contrast microscopy can be combined in order to detect different targets and structural properties in one sample.

One particularly important embodiment of the invention utilizes time-resolved fluorescence imaging using a conventional fluorescence microscope equipped with a pulsed excitation light source and a supplementary part containing a rotating aperture plate and an imaging detector (e.g. charge coupled device, CCD) as described in Soini et al. (Microsc. Res. Tech. 2003; 62:396-407). One of the advantages associated with time-resolved fluorescence imaging is that it allows discriminating between autofluorescence and specific fluorescence. The use of a time-domain together with labels having long lifetimes increases the sensitivity of detection as short-lived autofluorescence emitted by the object or the container itself has already decayed before the aperture plate lets the emission light, which now originates only from the label, pass to the CCD detector. Thus, improved contrast between the signal from the label and autofluorescence is achieved.

Another advantage associated with the time-resolved fluorescence imaging is that the fluorescence lifetime can be used to separate such fluorescent labels that cannot be distinguished by their fluorescence spectra. Thus, time-resolved fluorescence imaging enables increasing the number of labels used simultaneously at least by the factor of 3 compared to conventional fluorescence imaging.

Optionally, samples that are labelled with bioaffinity probes coupled with fluorescent or luminescent molecules may be analysed by photon counting (e.g. fluorometry) in addition to imaging. Such photon counting reveals the total amount of light emitted by the sample but no data on individual cells is obtained. According to the present invention, also time-resolved photon counting may be applied.

The final step of the method according to the present invention (e) is analysing more than one parameter in the sample. All parameters to be analysed may be parameters of the target in question. Alternatively, at least one of said parameters may be a parameter of a target and at least one of the other parameters to be analysed may be a parameter of a reference target. Also absence of a certain parameter of a target or a reference target is regarded as a parameter to be analysed. Said parameters may be analysed from one or more images.

When analysing the parameters involves counting, said counting is preferably computerized and done automatically by an image analysis software but can also be done with any other cell counting method known in the art.

In a preferred embodiment, the goal of the analysis is to determine the number of target cells as a proportion of e.g. total number of cells in the sample. Hence, the target cells, e.g. eosinophils or neutrophils, and the reference cells, e.g. all cells in the sample, are identified and counted. In such a case one analysed parameter is the number of eosinophils and the other analysed parameter is the total cell number. Such analysis may be used for measuring, diagnosing and screening airway diseases such as asthma, bronchitis and chronic obstructive pulmonary disease (COPD). As well known in the art, the proportion of eosinophils compared to other cells originating from lung is increased in the sputum of patients with asthma, whereas neutrophils are usually increased in the sputum of patients with COPD. However, eosinophils have also been found in some cases of COPD. Detection of the increased proportion of eosinophils and/or neutrophils in COPD patients may help predict the course of the disease and guide treatment decisions. The method according to the present invention allows early diagnosis of the disease (prior to severe respiratory symptoms) as well as monitoring the efficacy of therapy.

In another preferred embodiment, the method according to the present invention may be used to analyse foetal cells in maternal blood. Suitable foetal cell types for analysis include, but are not limited to, trophoblasts, lymphocytes, granulocytes and nucleated red blood cells. Foetal cells may be distinguished from maternal cells by any suitable way such as using specific antibodies. Foetal cells may then be subjected to chromosomal analysis revealing structural and/or numerical chromosome abnormalities. Said chromosomal analysis may be performed by any suitable method known in the art such as fluorescent in situ hybridisation (FISH) or its modifications. Thus, the method according to the present invention is suitable for prenatal screening or diagnosis.

In a particular embodiment, one parameter to be analysed is the foetal cell type and the other parameter is the chromosome number (either the total number or the number of certain chromosomes e.g. X, Y, 13, 18 or 21). On the other hand, one of the parameters to be analysed may also be the presence or the absence of a specific chromosomal marker.

The invention further relates to a kit for use in a method according to the present invention comprising a suitable container and at least one label for imaging desired targets in the sample. Preferably, the container is equipped with a means for draining liquid while retaining cells above a predetermined size. More preferably, the container is a compartment device, such as multi-well plate equipped with at least one filter having a pore size between 0.45 μm and 8 μm, preferably between 0.5 μm and 5 μm and even more preferably between 1 μm and 5 μm and still more preferably between 1 μm and 3 μm.

In one embodiment according to the present invention, the kit may further comprise one or more suitable reagents for pre-treating the sample, labelling the target and other reagents for specimen processing, washing and/or post-treatment purposes as described in more detail above. Such reagents may be provided in separate holders or they may be provided in the container. According to one preferred embodiment, all necessary reagents are provided in the container as a dried material that dissolves when appropriate reagents, optionally included in the kit, are added to the container.

In one preferred embodiment according to the present invention, the kit comprises a multi-well plate equipped with one removable prefiltering filter and one regular filter, reagents for solubization, permeabilization and fixation, one or more lanthanide chelates and/or a nuclear stain.

The above-described kits according to the present invention are suitable for prenatal diagnosing and screening chromosomal abnormalities; diagnosing and screening asthma; and diagnosing and screening COPD.

The following examples are given to further illustrate preferred embodiments of the present invention, but are not intended to limit the scope of the invention. It will be obvious to a person skilled in the art, as the technology advances, the inventive concept can be implemented in various ways. The invention and its embodiments are not limited to the examples described above but may vary within the scope of the claims.

### EXAMPLES

### Example 1

### Proportional Counting of Eosinophils in a Human Leukocyte Sample from Peripheral Blood Using Two Cytological Luminescent Stains

Human peripheral blood was taken into an EDTA tube (Venoject, Terumo, Leuven, Belgium). 100 μl of the sample was transferred into an eppendorf tube and pre-treated for 10 min with Optilyse™ according to manufacturer's recommendation in order to lyse red blood cells. Then the sample was centrifuged for 5 min at 400 g in room temperature and the supernatant was removed. Then 100 μl of PBS (Dulbecco) was added to the tube.

The resulting leukocyte suspension was transferred into a well of a 96-well filter plate (Acrowell, Pall Corporation, NY, USA). The liquid fraction of the sample was removed through the filter by vacuum-assisted aspiration. The suction was adjusted to enable draining of the solution through the filter and to concomitantly preserve the cell structures.

Next, 100 μl staining solution containing 2 μM europium chelate (PerkinElmer Life Sciences, Wallac Oy, Turku, Finland) for identification of eosinophils and 100 nM Syto 25 dye (Molecular Probes Inc., Eugene, Oreg., USA) in PBS for staining of all nuclei in the sample was added to the well. After 5 min incubation, the staining solution was discarded by aspirating through the filter. The cells were then washed twice by adding 200 μl of PBS (Dulbecco) to the well for 2 min and then removing the fluid by aspiration through the filter. Next, the cells were fixed with 3% glutaraldehyde (Ladd Research Industries; Burlington, Vt.) in PBS for 6 min followed by aspiration through the filter. The cells were washed with distilled water and then air-dried. The whole sample preparation was performed in room temperature.

Imaging and analysis: The specimen was imaged under a fluorescent microscope equipped with an arrangement for time-resolved imaging as described above. Imaging was performed with a 10× objective (Planfluor, Nikon, Japan). The camera was adjusted to record over several excitation pulses for 0.5 to 5 seconds depending on the brightness of the signal while the rotating aperture plate opened the beam of light to the camera during or a predetermined time after every excitation pulse. Open-close cycles of the rotating aperture plate were synchronized with the excitation pulses so that the close-time after each pulse was 200 μs and the open-time 600 μs.

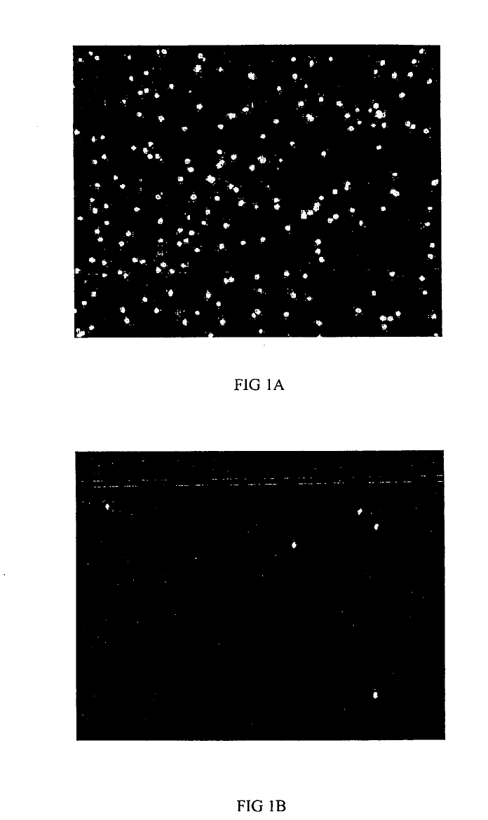

The image of the fluorescence signal from Syto 25 was recorded using the filter set G-2A (Nikon, Japan) (FIG. 1). The time delay was adjusted to record the image simultaneously when the specimen was excited.

The image of the fluorescence signal from Eu was recorded using the filter set consisting of an excitation filter RUG11 (Schott, Germany), a dichroic mirror DM400 (Nikon, Japan) and an emission filter 615/7.5 nm (Barr Associates Inc, USA). Eu-image was recorded with 300 μs delay adjustment. The resulting image can be seen in FIG. 2.

The above arrangement assured that the signal from Syto 25 stain having a broad emission spectrum did not interfere with the signal from Eu.

The number of positive cells in each image was then counted with Image analysis software (Image Pro Plus 4.5, Media cybernetics, USA). The image contained 196 cells, 5 of which were eosinophils.

### Example 2

### Determination of the Proportion of Eosinophils and Neutrophils in a Peripheral Blood Leukocyte Sample Using a Luminophore-Labelled Antibody Together with Two Cytological Stains

The specimen is human peripheral blood taken into a EDTA tube (Venoject, Terumo, Leuven, Belgium). 100 μl of blood is transferred into a 6 ml test tube. Cell membranes are stabilized and permeabilized by incubating the cells for 2 min with 2 ml of Ortho Permeafix™ according to the manufacturer's recommendation. Next the sample is centrifuged for 20 min at 400 g and 20 degrees C. The supernatant is removed. The sample is then washed with a solution of Na2HPO4 (10 mM), NaCl (150 mM), KCl (4 mM), EDTA (200 mM), 1.5% bovine serum albumin (BSA, Pharmacia Amersham, Uppsala, Sweden), 5% fetal calf serum (FCS, titrated to pH 7.4 with HCl, Autogen Bioclear, Wiltshire). After 10 min the sample is centrifuged for 5 min at 400 g in room temperature and supernatant is removed. Finally 100 μl PBS (Dulbecco) is added into the tube.

The pre-treated sample is then transferred into 96 well multi-well filter plate as described in Example 1 above.

100 μl of incubation solution containing 0.02 mg/ml terbium-labelled anti-human neutrophil lipocalin (HNL) antibody, (Pharmacia, Uppsala, Sweden) is then added to the well for identification of neutrophils. After 30 min incubation, the well is drained through the filter. Next, the well is washed twice by adding and subsequently removing 100 μl PBS.

Eosinophils and all the nuclei in the sample are stained as described in Example 1 above.

Sample imaging and analysis is performed essentially as described in Example 1 and Example 4.

### Example 3

### Method for Detecting the Proportion of T-Cell Lymphocytes and Eosinophils in a Peripheral Blood Leukocyte Sample Using a Long-Life Luminophore-Labelled Antibody Against a Cell-Surface Marker and Two Cytological Stains Against Intracellular Markers

Human peripheral blood is taken into an EDTA tube (Venoject, Terumo, Leuven, Belgium). Then 100 μl of the sample is transferred into an eppendorf tube and treated for 5 min with 8 μl of Tb-labeled anti-CD3 antibody (obtainable from HyTest (Turku, Finland) labelled by PerkinElmer Life and Analytical Sciences (Turku, Finland)). The sample is then treated for 10 min with Optilyse™ according to manufacturer's recommendation in order to lyse red blood cells. Next, the sample is centrifuged for 5 min at 400 g in room temperature, the supernatant is removed and 100 μl of PBS (Dulbecco) is added to the tube.

The resulting leukocyte suspension is transferred into a well of a 96-well filter plate as described in Example 1.

Eosinophils and all the nuclei in the sample are stained as described in Example 1 above.

The specimen is imaged and analysed essentially as described in Example 1 and 4.

### Example 4

### Method for Detecting the Proportion of Eosinophils in a Sputum Sample Using Two Cytological Stains

Samples of induced sputum were obtained from voluntary subjects suffering from severe asthma. Sputum production was induced by inhalation of 5 ml of 3% NaCl solution from an ultrasonic nebulizer (Omron U1, Omron, Germany) as described in Metso et al. (1996).

The sputum sample was examined under an inverted microscope, and viscous parts were collected with forceps. Next, 20 mg of viscous samples were placed into wells of a prefiltering microtitre plate equipped with a filter having a pore size of 40 μm. The mucus was solubilized with a four-fold volume of 6.5 mmol/l dithiotreitol (Sputolysin™, CalbioChem, LaJolla, USA; diluted ten-fold with distilled water). The sputum specimen was incubated for 15 minutes in the microtitre plate on a microtitre plate mixer. 100 μl of PBS was added into the well and incubation was continued for another 5 minutes. The sputum specimen in Sputolysin™ was passed through the filter and was collected into a well of a processing and imaging plate equipped with a filter having a pore size of 1.2 μm. The prefiltering plate was then removed.

Sputum cell suspension specimen was rinsed twice in PBS (by adding 250 μl PBS, waiting for 1 min, and emptying the well through the filter between the rinsing cycles by vacuum). The membranes of the sputum cells were stabilized and permeabilized with 250 μl of concentrated Permeafix™ in the well of the processing and imaging plate. After fixation and permeabilization the specimens were rinsed as described above followed by staining in a mixture of eosinophil specific stain consisting 2 μM solution of inactivated terbium chelate and of Syto 25 nuclear stain. The specimen was rinsed in PBS as described above and postfixed in 2.5% glutaraldehyde in PBS, rinsed in water and then dried.

The specimen was imaged and analysed as described in Example 1 above. Additionally, the image of the fluorescence signal from Tb was recorded using the filter set consisting of an excitation filter RUG11 (Schott, Germany), a dichroic mirror DM400 (Nikon, Japan) and an emission filter 545/7.5 nm (Barr Associates Inc, USA). Tb-image was recorded with 300 μs delay adjustment. This arrangement assured that the signal from Syto 25 stain having a broad emission spectrum did not interfere with the signal from Eu or Tb.

The image contained 554 cells, 2 of which were eosinophils.

1. A method for analysing a mixed biological sample by imaging, comprising the steps of
a) pre-treating a sample obtained from a subject;
b) transferring said sample into a container;
c) processing said sample;
d) detecting at least one target in said sample by imaging and
e) analysing more than one parameter,
wherein steps c)-d) are performed in a single container equipped with at least one filter.

2. The method according to claim 1, wherein steps a)-d) are performed in a single container, and step b) is performed prior to step a).

3. The method according to claim 1, wherein step a) comprises target protection, homogenization, organel extraction and/or removing a certain cell population.

4. The method according to claim 3, wherein said target protection is performed using EDTA and/or citrate.

5. The method according to claim 1, wherein step c) comprises fixation, permeabilization, labelling of the target and/or post-fixation.

6. The method according to claim 5, wherein said labelling of the target is performed using a long decay time photoluminescent label.

7. The method according to claim 1, wherein step d) is performed by bright field, dark field, fluorescence, or time-resolved fluorescence imaging.

8. The method according to claim 7, wherein step d) is further performed by fluorometry.

9. The method according to claim 1, wherein said container is a multi-well device.

10. The method according to claim 9, wherein said filter has a pore size from about 0.45 μm to about 8 μm.

11. The method according to claim 10, wherein said filter has a pore size from about 0.5 μm to about 5 μm.

12. The method according to claim 11, wherein said filter has a pore size from about 1 μm to about 5 μm.

13. The method according to claim 12, wherein said filter has a pore size from about 1 μm to about 3 μm.

14. The method according to claim 1, wherein said mixed biological sample comprises granulocytes, inflammatory cells, cancer cells, cells from immunodefence system and/or foetal cells.

15. The method according to claim 1, wherein said mixed biological sample is a body fluid, a lavage fluid, a tissue homogenate, an expectoration sample, an excretion, an aspiration sample, a biopsy sample or a cell culture.

16. The method according to claim 1, wherein step e) is performed by computerized image analysis.

17. A method for prenatal diagnosing or screening chromosomal abnormalities by analysing foetal cells in maternal blood using a method according to claim 1.

18. A method for diagnosing or screening asthma by analysing the proportional number of eosinophils in sputum using a method according to claim 1.

19. A method for diagnosing or screening chronic obstructive pulmonary disease by analysing the proportional number of eosinophils and/or neutrophils in sputum using a method according to claim 1.

20. A kit for imaging a mixed biological sample in a method according to claim 1, comprising a container and at least one labelling agent for detecting the target, wherein said container is equipped with a means for draining liquid while retaining at least some of the cells in said sample.

21. The kit according to claim 20 further comprising at least one removable filter.

22. The kit according to claim 20, wherein said container is a multi-well plate.

23. The kit according to claim 20 comprising necessary reagents for performing pre-treatment and processing of said sample.

24. The method according to claim 15, wherein step e) is performed by computerized image analysis.

25. A method for prenatal diagnosing or screening chromosomal abnormalities by analysing foetal cells in maternal blood using a method according to claim 16.

26. A method for diagnosing or screening asthma by analysing the proportional number of eosinophils in sputum using a method according to claim 16.

27. A method for diagnosing or screening chronic obstructive pulmonary disease by analysing the proportional number of eosinophils and/or neutrophils in sputum using a method according to claim 16.

28. A kit for imaging a mixed biological sample in a method according to claim 16, comprising a container and at least one labelling agent for detecting the target, wherein said container is equipped with a means for draining liquid while retaining at least some of the cells in said sample.
